(19) United States
(12) Patent Application Publication
(10) Pub. No.: US 2015/0066119 A1
(43) Pub. Date: Mar. 5, 2015

(54) INFANT WARMING SYSTEMS

(71) Applicant: Embrace Innovations, San Francisco, CA (US)

(72) Inventors: Rahul A. PANICKER, Bangalore (IN); Eu-wen DING, Singapore (SG); Srishti SUNDRAM, Bhagalpur (IN); Satyan RAJ, Bangalore (IN); Honey BAJAJ, Pin (IN); Jane CHEN, San Francisco, CA (US); Naganand MURTY, Bangalore (IN); Linus LIANG, Saratoga, CA (US)

(73) Assignee: Embrace Innovations, San Francisco, CA (US)

(21) Appl. No.: 14/013,914

(22) Filed: Aug. 29, 2013

(51) Int. Cl. A61F 7/08; A61F 7/00

(52) U.S. Cl. CPC A61F 7/08; A61F 7/0085; A61F 2007/0095
USPC 607/104; 607/96; 607/114

(57) ABSTRACT

Infant warming systems are described herein generally comprising a temperature regulation assembly which encloses a phase change material (PCM). The temperature regulation assembly may be initially heated (or re-heated) by water contained within a heating assembly and then placed within a retaining pouch of an infant bedding. The PCM may then provide constant heat over a period of time to the infant. A control/indicator interface coupled to the regulation assembly may constantly monitor the temperature of the PCM as well as the ambient temperature to provide one or more indications or alerts to the user with respect to one of several states of the warming assembly.

### FIELD OF THE INVENTION

The present invention relates to medical devices. In particular, the present invention relates to methods and apparatus for facilitating and monitoring infant warming systems. More particularly, the present invention relates to methods and apparatus for facilitating and monitoring infant warming systems which utilize phase change materials for providing and maintaining an elevated temperature to an infant.

### BACKGROUND OF THE INVENTION

Babies who are born prematurely, with low-birth-weight (LBW), or weak and ill-developed, may have a particularly difficult time regulating body temperature because their nervous regulating mechanisms are often underdeveloped. If heat loss is not prevented and is allowed to continue, such babies will develop hypothermia. A hypothermic baby, especially if it is small, sick, or is of LBW, is at increased risk of developing health problems and of dying.

Twenty million premature and LBW babies susceptible to thermoregulation problems are born every year around the world. In developed countries, LBW babies are placed in incubators to regulate their temperature. Typical incubators are very expensive, costing thousands of US dollars. Such incubators require active electrical connections and may require delicate electronics.

Various warming devices may be utilized to warm infants and particularly newborn infants in order to maintain the infant's body temperature within a predetermined range. Such infant warmers commonly radiant heaters which radiate infrared energy upon the infant to maintain the predetermined temperature. However, such conventional infant warmers rely on a constant source of electricity in order to remain operational yet in many regions of the world, such as developing countries, such electrical sources may be unavailable or unreliable.

Other devices have included the use of electric blankets which incorporate resistive heating elements in order to produce the desired heat. Yet such electric blankets not only require a similar electrical source but also fail to distribute heat evenly and may further require sophisticated electronics to regulate heat.

While other devices have been used which utilize heat exchange materials for regulating body temperature, such devices have been unsuitable for use in heating and/or maintaining the body temperature for infants. This is particularly the case since infants have a relatively narrow temperature range in which their bodies should be maintained.

Hence, there remains a need for improved thermal regulation apparatus and methods that address the problems associated with existing thermal regulation designs.

### SUMMARY OF THE INVENTION

An infant warming assembly which utilizes a phase change material (PCM) may be used to warm the infant without the need for electricity. Such a system may comprise a temperature regulation assembly for regulating the temperature of an infant, generally comprising a phase change material which changes between a liquid phase and a solid phase within a predetermined temperature range, at least one temperature sensor in thermal communication with the phase change material, and a control/indicator interface in communication with the at least one temperature sensor, the interface being programmed to provide an indication or alert to a user when the phase change material is within the predetermined temperature range.

In use, the system may generally implement methods of regulating temperature of an infant by providing a temperature regulation assembly having a phase change material which changes between a liquid phase and a solid phase within a predetermined temperature range, positioning the temperature regulation assembly within a retaining pouch of a heating assembly having an adjacent reservoir filled with a heated volume of water, providing an indication or alert when a temperature of the temperature regulation assembly is between 35° to 41° C., and further positioning the temperature regulation assembly within a retaining pouch of a bedding such that the temperature of the infant in contact with the bedding is regulated.

Further examples of an infant warmer utilizing a PCM are shown in further detail in U.S. Pat. No. 8,257,417; U.S. Pub. 2012/0305231; and U.S. 201210330388, each of which is incorporated herein by reference in its entirety and for any purpose. Moreover, various features shown and described in each of the incorporated references may be combined in any number of combinations with the various features disclosed herein.

One variation of the infant warming assembly may generally comprise a temperature regulation assembly to which a control/indicator interface may be attached for monitoring various parameters of the assembly. The temperature regulation assembly (once suitably heated) may be positioned within a retaining pouch defined along a bedding which may optionally include one or more straps for securing the infant. The retaining pouch defined in the bedding may separate the temperature regulation assembly from the infant but maintains thermal coupling with the infant. An additional bed sheet (e.g., fabricated from cotton) may also be optionally placed around or upon the bedding for contact against the infant's body to prevent any skin reaction from being induced upon the infant's body.

When the newborn is placed upon the bedding, the infant warming assembly provides warmth such that a warm microclimate is created around the newborn while also providing the newborn warmth through dorsal thermal conduction from the temperature regulation assembly. Once heated, the infant warming assembly may maintain a temperature of about, e.g., 37° C., for about, e.g., 8 hours, without needing to be re-heat the temperature regulation assembly. Moreover, such a warming assembly may be used in a variety of ambient conditions varying from a temperature range of, e.g., 10° to 34° C., with a humidity range of, e.g., 30% to 75%.

The heating assembly may incorporate a fluid reservoir having a port into which a heated fluid, e.g., 2.7 to 3 liters of boiling water, may be poured into. A funnel may be optionally positioned within or in proximity to the port for facilitating the introduction of the water into the reservoir. It is this heated water retained within the reservoir which serves as the source of warmth to elevate the temperature regulation assembly to its operating temperature. Once the reservoir is filled, the temperature regulation assembly may be positioned adjacent to the reservoir and separated but still in thermal communication. An interface attachment, such as a flap, may extend from the heating assembly and connect or couple to the control/indicator interface to optionally silence any alarms or alerts on the interface. With the assembly suitably positioned and retained within the retaining pouch, the heating assembly may be laid on a flat surface to allow the PCM contained within the assembly to absorb the heat via thermal conduction and/or convection from the heated water in the adjacent reservoir.

Under normal operating conditions, the temperature regulation assembly 12 may take, e.g., about 45 minutes, to heat within the heating assembly 30 to a suitable operating temperature for use. However, if the assembly is re-heated after use, it may take about, e.g., 20 minutes or less, to re-heat back to its desired operating temperature.

When the temperature regulation assembly 12 has reached its operating temperature, the control/indicator interface may indicate through a visual and/or auditory alarm (e.g., changing from a flashing red light to a flashing green light and/or with a beeping alert) that the assembly is ready for use. The temperature regulation assembly may then be removed from the heating assembly and inserted into the retaining pouch of the bedding for use.

The bedding itself may be insulated to minimize heat loss from the temperature regulation assembly. The one or more straps extending over the bedding may be adjustably tightened or loosened, e.g., via hook-and-loop fasteners, hooks, buttons, etc., around the infant for ensuring close contact with the infant body as well as securing the infant in place. The top surface of the bedding may be made of a water-resistant material that facilitates cleaning of the bedding. Moreover, bunting may be provided around the periphery of the bedding to further prevent the infant from rolling off the bedding while still allowing for complete access and visibility to the parent or care provider. Additionally, the open design of the bedding further allows for close parent-to-child interaction even when placed upon the bedding. Moreover, the bedding may further ensure that the heat provided by the temperature regulation assembly is dispersed and evenly distributed over the bedding to the infant and may further ensure that the temperature regulation assembly stays warm for about, e.g., 8 hours or more.

Aside from the PCM material, the assembly may also comprise an enclosed layer of water which is positioned atop of the PCM to provide for further support to the infant as well as to ensure that the heat and temperature is uniformly distributed over the assembly. The assembly may be comprised of a retaining tray which defines a PCM retaining portion surrounded by a retaining edge or lip around the periphery of the tray. One or more temperature sensors, e.g., thermistor, thermocouple, etc., may be positioned along the retaining tray to monitor the temperature of the PCM.

The retaining tray may provide a retaining channel or depression for the temperature regulation element which may enclose the PCM. The PCM may generally comprise a substance which melts or solidifies at a particular temperature or temperature range thereby storing or releasing energy at that particular temperature or range. During a phase change from liquid to solid or vice versa, the energy absorbed or released by the PCM is used to facilitate the phase change of the material and thus the temperature of the PCM remains constant. The PCM preferably has a high heat of fusion which allow it to store and release large amounts of energy. Additionally, the PCM may comprise either an organic or inorganic material, e.g., n-eicosane, Tetradecanol, eutectic mixtures, or any other suitable material.

While the temperature regulation element may continue to provide heat at its operating temperature (e.g., for about 8 hours or more at an ambient temperature of about 25° C.), the heat-providing capacity of the temperature regulation element may depend on factors such as weight and health of the newborn as well as operating conditions such as ambient temperature, as described in further detail below. With the temperature regulation element so positioned within retaining portion, a separate tray covering may be secured to the retaining edge or lip around the periphery of the tray to fully enclose the temperature regulation element within the tray.

With the temperature regulation element fully enclosed, a separate water layer retained within an enclosure may then be secured upon the tray covering. The water layer may provide for further support to the infant as well as to ensure that the heat and temperature is uniformly distributed over the assembly. The fully assembled temperature regulation assembly may accordingly be heated (and re-heated as needed) within the retaining pouch of heating assembly and then transferred to the retaining pouch of the bedding. Once the temperature regulation assembly is placed within bedding for use with an infant, the PCM within temperature regulation element may provide for warming heat for up to 8 hours or more.

Turning now to the control/indicator interface, the interface may comprise a processor and associated electronics within which continuously monitor both the temperature of the temperature regulation assembly as well as the ambient temperature. In the event that the various temperatures are too high or too low for any reason, the interface may provide a visual and/or auditory indicator or alarm to alert the user to take corrective action, if needed, such as when the temperature regulation assembly is safe to use with an infant (e.g., a green light may flash) or when the temperature regulation assembly is unsafe due to high or low temperatures (e.g., a red light may flash with or without an auditory alarm). The indicator may be powered by an on-board power supply, e.g., batteries, or any other type of power supply.

The control/indicator interface may also incorporate memory storage to collect and store various forms of data monitored and sensed by the system or user as well as a communication interface to transfer the data (e.g., wired or wirelessly) to or from an external device.

The indicator may also be provided with one or more actuation features, such as a button, touchscreen, etc. to place the processor within the indicator within one of several available functions. A first function may silence or “snooze” any visible and/or audible alarms for about, e.g., 1.5 minutes, although this period of time may be programmed to be more or less. A second function of the indicator may be used when the assembly is in use with an infant. When activated, a PCM charge indication feature may allow the user to estimate how long the assembly will last so that the user may plan accordingly. Finally, a third function may initiate a demonstration mode after which the indicator may revert to its normal state.

Because of the differences between the temperature of the assembly and the variability of the ambient temperature, the processor within indicator may be programmed to follow one of several different algorithms to ensure safety to the infant. The indicator may be programmed to distinguish when the assembly is positioned inside the receiving pouch of the heating assembly (e.g., when the interface attachment is coupled to the indicator). Similarly, the indicator may also be programmed to distinguish when the assembly is positioned outside of the heating assembly such as when the unit is simply removed from the heating assembly or when the unit is placed within the receiving pouch of the bedding for use as shown by the various states within block.

When the assembly is positioned within the heating assembly, it may comply with one of several different states. For instance, prior to or during heating of the assembly, the assembly may be indicated as being in a COLD state, e.g., by showing a red light and remaining silent, until its temperature is sensed by indicator as being below 36.0° C. In the event its temperature rises to be greater than or equal to 36.5° C. indicator may enter an OK TO USE state, e.g., by showing a green light and alerting with a Medium Priority alarm. However, if the assembly becomes overheated and unsafe for use, indicator may enter an OVERHEATED state, e.g., by showing a red light and alerting with a High Priority alarm (which may or may not be optionally silenced). The assembly may be considered overheated if the sensed temperature is either greater than or equal to 41° C. or if the sensed temperature is greater than or equal to 38.5° C. along with a temperature rate change over time (dT/dt) of 0.2 degrees/30 seconds is also detected. However, if the temperature falls below 40.5° C. along with a negative temperature rate change over time, then the indicator may revert to its OKAY TO USE state.

Because use of the assembly may be affected by the ambient temperature, the indicator may also be configured to sense the ambient temperature as well and account for this variable condition. For instance, in the event that a HIGH AMBIENT state is detected (e.g., when the ambient temperature is greater than or equal to 32° C.), the indicator may show a red light yet remain silent if the assembly is still positioned within the heating assembly. If the temperature of the assembly is sensed to be greater than or equal to 41° C., then the indicator may enter the OVERHEATED state. Otherwise, if the ambient temperature is less than or equal to 32° C. and the detected ambient temperature remains so for greater than 2 minutes, then the indicator may enter into a COLD or OK TO USE state. The 2 minute waiting period may be programmed into the indicator to ensure that any temporary temperature fluctuations are dampened and accounted for although this waiting period may be less than or greater than 2 minutes or may be omitted entirely if so desired.

In the event that the temperature regulation assembly has been removed from the heating assembly, e.g., for use within the bedding, then the indicator may indicate or alert any one of the states shown in block. For instance, in the event that a temperature of the assembly is less than 35° C., a COLD state may be indicated, e.g., by showing a red light and indicating a High Priority alarm, to alert the user that the assembly requires heating and should not be used with an infant.

However, if the assembly is sensed to fall below 35.3° C. during its use, then an EARLY HEATING INDICATION state may be indicated, e.g., by showing a green light and alerting with an optional repeating alert or alarm, to alert the user that the assembly may need to be re-heated within a predetermined period of time, e.g., within 30 minutes, to allow the user to make preparations accordingly such as by heating water for the heating assembly. If the assembly is sensed to further fall below 35.0° C., then it may re-enter the COLD state; otherwise, if the assembly is sensed to rise to be greater than or equal to 36° C., then the indicator may enter an OK TO USE state. However, in the event that the assembly is sensed to rise in temperature to be greater than or equal to 41° C. then the indicator may enter an OVERHEATED state, e.g., by showing a red light and alerting with a High Priority alarm, to alert the user that the assembly should not be used with the infant and should be allowed to cool before use. If the indicator senses that the temperature has fallen below 38.5° C. then the indicator may enter back into its OK TO USE state.

Because use of the assembly may be affected by the ambient temperature as sensed by the indicator, an additional state may be accounted for as shown by the HIGH AMBIENT state. Additionally, with the various states (when the assembly is either within the heating assembly or outside the heating assembly), the temperature regulation assembly may be re-positioned between the heating assembly and bedding (or simply removed from the heating assembly).

Additionally and/or alternatively, the system may also incorporate a number of features to further provide for the safety and comfort of the infant placed within the system. For instance, the control/interface unit may alert to conditions such as the ambient temperature being too low or too high or it may automatically trigger other actions that ensure usage of the device when in a safe ambient temperature range. Moreover, the system may also incorporate one or several monitoring peripherals to monitor various physiological parameters of the infant (e.g., vital parameters such as infant temperature, weight, oxygen levels, respiration, heart rate, etc.). Additionally, any number of monitoring and/or peripheral devices, e.g., air-purifiers, may be used in combination with the system and optionally connected to the control/indicator interface (e.g., wired or wirelessly). Such devices may be used to help create or maintain hygienic levels of air quality (in one example) around the infant. Other devices may be used in other examples such as ambient warmers, humidity controllers, etc.

### BRIEF DESCRIPTION OF THE DRAWINGS

FIGS. 1A and 1B show top and perspective assembly views of one variation of an infant warming assembly utilizing several components.

FIG. 2 shows a perspective assembly view of the several components which may be utilized in the infant warming assembly.

FIG. 3 shows a perspective view of an infant bedding having an optional retaining strap and pouch for receiving a temperature regulation assembly.

FIG. 4 shows a perspective view of one variation of a temperature regulation assembly having a control/indicator interface for monitoring a temperature of the assembly.

FIGS. 5A to 5C show perspective, top, and side views of a retaining tray for holding a temperature regulation element.

FIG. 5D shows a perspective view of a retaining tray with a temperature regulation element positioned within the tray.

FIG. 5E shows a top view of a tray covering which may be attached to the retaining tray for maintaining a position of the temperature regulation element.

FIGS. 5F and 5G show perspective views of a temperature regulation assembly prior to assembly with the control/indicator interface and with the interface attached.

FIG. 6 shows a perspective view of a heating assembly which may be used to heat the temperature regulation assembly.

FIG. 7 shows a partial cross-sectional top view of a reservoir which may be contained within the heating assembly for retaining a heated fluid such as water for heat transfer to the temperature regulation assembly.

FIG. 8 shows a top assembly view of the heating assembly having the temperature regulation assembly contained within for heating of the temperature regulation element.

FIG. 9 shows a graph of the temperature change over time of the temperature regulation element.

FIG. 10 shows a chart illustrating examples of the various types of indications and/or alerts which may be displayed by the control/indicator interface.

FIG. 11 shows a flow diagram of the various heating and regulation modes of the temperature regulation assembly which may be monitored by the control/indicator interface.

FIG. 12 shows the diagram of FIG. 11 illustrating the various states between which the temperature regulation assembly may be re-positioned.

FIG. 13 shows a top view of an infant placed within a heating assembly and an optional monitoring peripheral attached to the infant and control/indicator interface.

FIG. 14 shows a top view of an infant placed within the heating assembly and an optional monitoring and/or peripheral device in communication with the control/indicator interface.

### DETAILED DESCRIPTION OF THE INVENTION

In elevating and maintaining the temperature of an infant, particularly of a low birth weight newborn infant weighing, e.g., 1.5 to less than 2.5 kilograms, an infant warming assembly which utilizes a phase change material (PCM) may be used to warm the infant without the need for electricity. Such a system may be effective and simple enough to use by parents at the infant's home or in a clinical environment by medical staff and in settings where electrical power may be unavailable or uncertain. Because electrical power is not needed to heat and maintain the phase change material at an elevated temperature, the possibility of burns near the infant are also eliminated.

Further examples of an infant warmer utilizing a PCM are shown in further detail in U.S. Pat. No. 8,257,417; U.S. Pub. 2012/0305231; and U.S. 2012/0330388, each of which is incorporated herein by reference in its entirety and for any purpose. Moreover, various features shown and described in each of the incorporated references may be combined in any number of combinations with the various features disclosed herein.

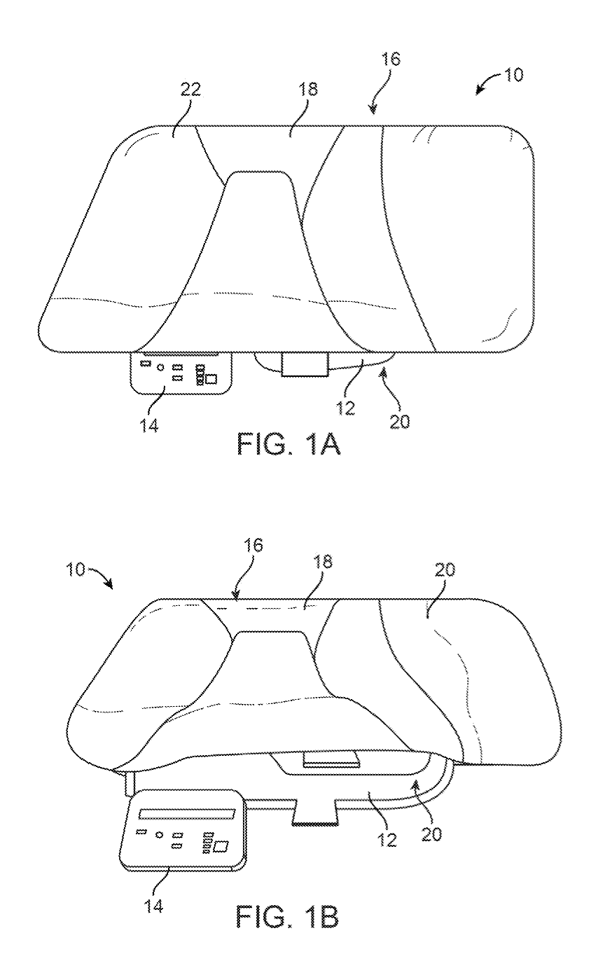

One variation of the infant warming assembly 10 may generally comprise a temperature regulation assembly 12 to which a control/indicator interface 14 may be attached for monitoring various parameters of the assembly 12. The temperature regulation assembly 12 (once suitably heated) may be positioned within a retaining pouch 20 defined along a bedding 16 which may optionally include one or more straps 18 for securing the infant, as shown in the top view of FIG. 1A. The retaining pouch 20 defined in the bedding 16 may separate the temperature regulation assembly 12 from the infant but maintains thermal coupling with the infant. FIG. 1B shows a perspective assembly view of the warming assembly 10 having the temperature regulation assembly 12 partially removed from the retaining pouch 20 of bedding 16 to illustrate the insertion and positioning of the assembly 12 within the bedding 16. An additional bed sheet 22 (e.g., fabricated from cotton) may also be optionally placed around or upon the bedding 16 for contact against the infant's body to prevent any skin reaction from being induced upon the infant's body. Because the bed sheet 22 is replaceable, cleaning of the warming assembly 10 is further simplified.

When the newborn is placed upon the bedding 16 with the bed sheet 22 and is covered with a blanket, the infant warming assembly 10 provides warmth such that a warm microclimate is created around the newborn while also providing the newborn warmth through dorsal thermal conduction from the temperature regulation assembly 12. Once heated, the infant warming assembly 10 may maintain a temperature of about, e.g., 37° C. for about, e.g., 8 hours, without needing to be reheat the temperature regulation assembly 12 (which is the same temperature range as recommended by the World Health Organization for a heated water mattress). Moreover, such a warming assembly 10 may be used in a variety of ambient conditions varying from a temperature range of, e.g., 10^{°} to 34° C. with a humidity range of e.g., 30% to 75%.

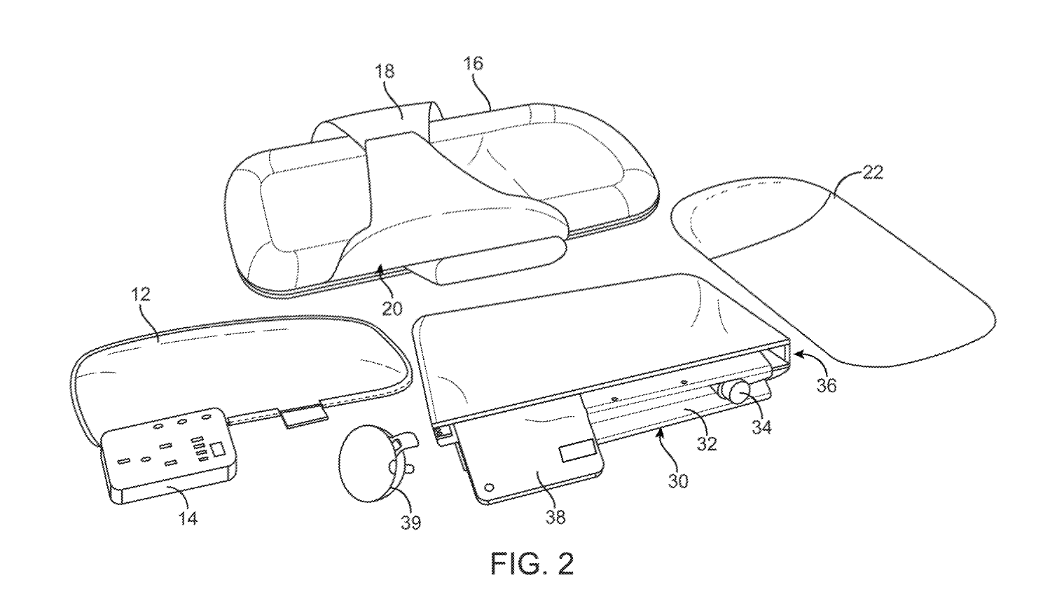

FIG. 2 shows a perspective assembly view of the various components which may be used in operating the infant warming assembly 10. As shown, the removable bed sheet 22 is shown removed from the bedding 16. Also, the temperature regulation assembly 12 is also shown removed from the retaining pouch 20 of bedding 16. Additionally, a variation of the heating assembly 30 is shown which may be utilized to heat the temperature regulation assembly 12 to an elevated temperature prior to insertion within the retaining pouch 20 of bedding 16 for use with the infant. The heating assembly 30 may incorporate a fluid reservoir 32 having a port 34 into which a heated fluid, e.g., 2.7 to 3 liters of boiling water, may be poured into. A funnel 39 may be optionally positioned within or in proximity to the port 34 for facilitating the introduction of the water into the reservoir 32. The funnel 39 may further define a visual indicator such as a marking or graduation along the funnel as a fill indicator to provide the user a visual indication as to when the reservoir 32 is sufficiently filled with the heated water.

It is this heated water retained within the reservoir 32 which serves as the source of warmth to elevate the temperature regulation assembly 12 to its operating temperature. Once the reservoir 32 is filled, the temperature regulation assembly 12 may be positioned adjacent to the reservoir 32 and separated but still in thermal communication. An interface attachment 38, such as a flap, may extend from the heating assembly 30 and connect or couple to the control/indicator interface 14 to optionally silence any alarms or alerts on the interface 14. With the assembly 12 suitably positioned and retained within the retaining pouch 36, the heating assembly 30 may be laid on a flat surface to allow the PCM contained within the assembly 12 to absorb the heat via thermal conduction and/or convection from the heated water in the adjacent reservoir 32.

Under normal operating conditions, the temperature regulation assembly 12 may take, e.g., about 45 minutes, to heat within the heating assembly 30 to a suitable operating temperature for use. When the temperature regulation assembly 12 has reached its operating temperature, the control/indicator interface 14 may indicate through a visual and/or auditory alarm (e.g., changing from a flashing red light to a flashing green light and/or with a beeping alert) that the assembly 12 is ready for use, as described in further detail below. The temperature regulation assembly 12 may then be removed from the heating assembly 30 and inserted into the retaining pouch 20 of bedding 16 for use.

If the ambient environment is exceptionally cold, it can take about, e.g., 1 hour or more, for the heating temperature regulation assembly 12 to heat the temperature regulation assembly 12 to its suitable operating temperature. However, during continuous use it may take only, e.g., about 15 to 20 minutes or less, to reheat the temperature regulation assembly 12 that has only recently started to indicate an overcooled state, as described in further detail below.

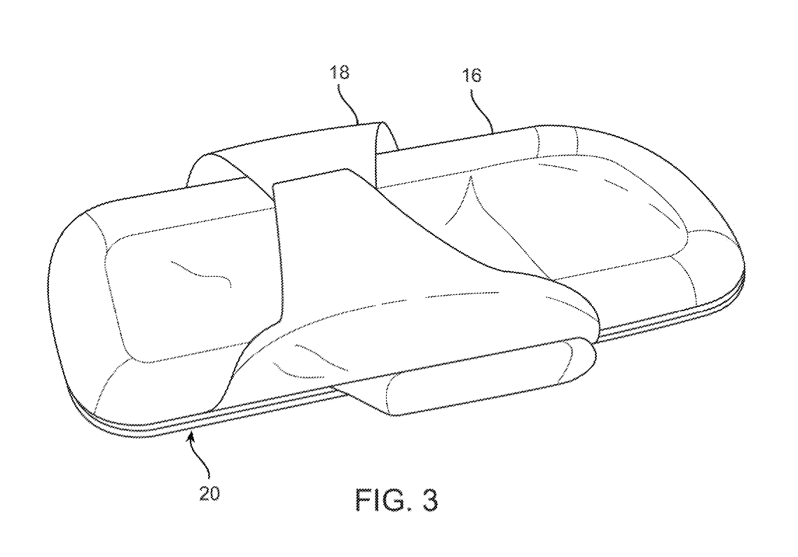

FIG. 3 shows a perspective view of the bedding 16 with the retaining pouch 20 into which the temperature regulation assembly 12 may be inserted. The bedding 16 itself may be insulated to minimize heat loss from the temperature regulation assembly 12. The one or more straps 18 extending over the bedding 16 may be adjustably tightened or loosened, e.g., via hook-and-loop fasteners, hooks, buttons, etc., around the infant for ensuring close contact with the infant body as well as securing the infant in place. The top surface of the bedding 16 may be made of a water-resistant material that facilitates cleaning of the bedding 16. Moreover, bunting may be provided around the periphery of the bedding 16 as shown to further prevent the infant from rolling off the bedding 16 while still allowing for complete access and visibility to the parent or care provider. Additionally, the open design of the bedding 16 further allows for close parent-to-child interaction even when placed upon the bedding 16. Moreover, the bedding 16 may further ensure that the heat provided by the temperature regulation assembly 12 is dispersed and evenly distributed over the bedding 16 to the infant and may further ensure that the temperature regulation assembly 12 stays warm for about, e.g., 8 hours or more.

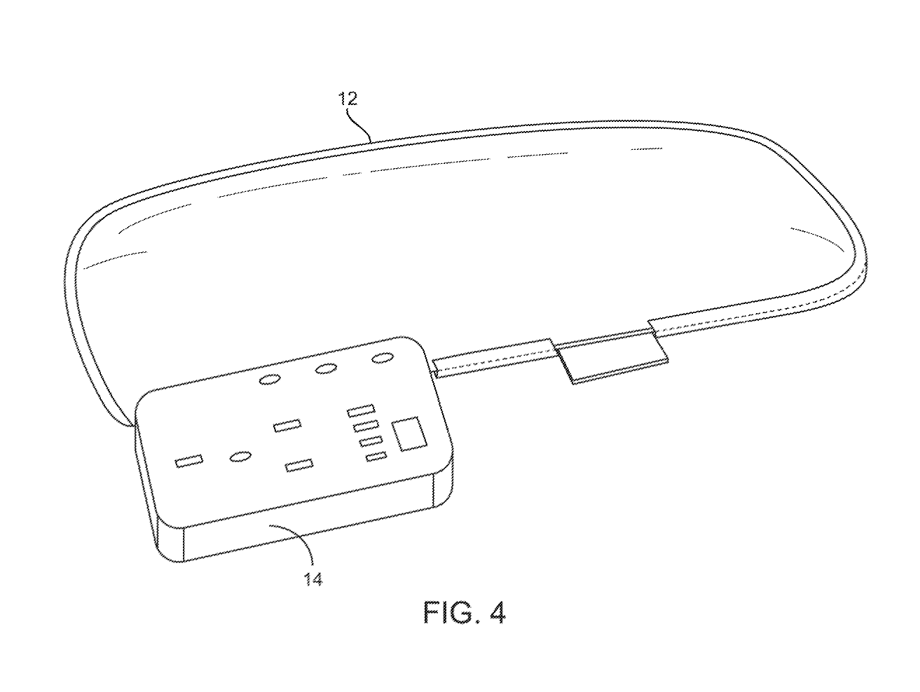

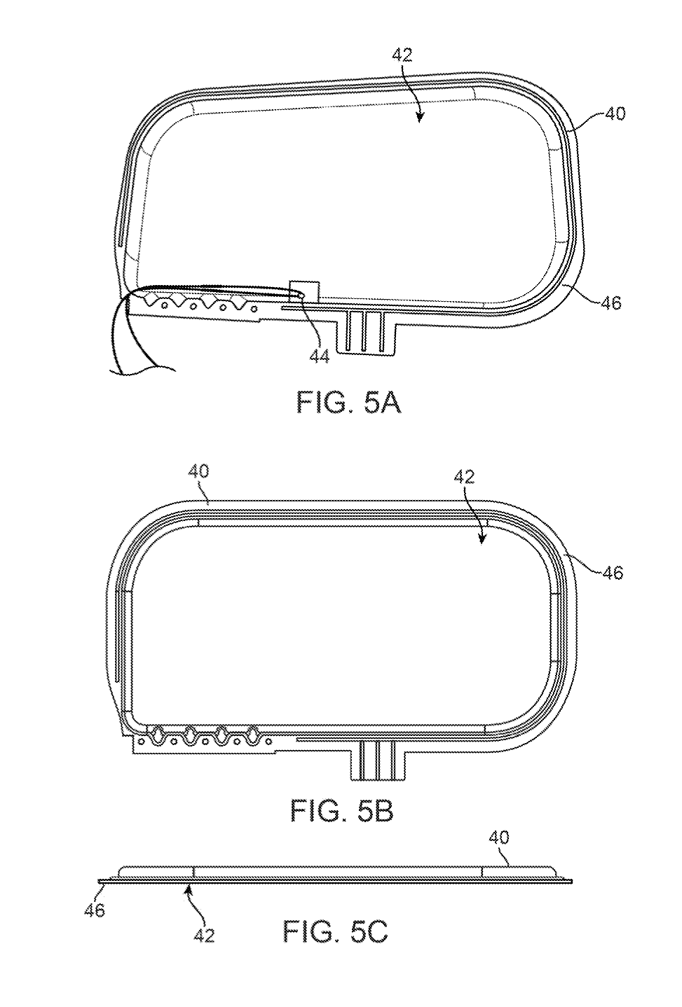

Turning now to the temperature regulation assembly 12 itself, the assembly 12 and the attached interface 14 may be seen in the perspective view of FIG. 4. Aside from the PCM material, assembly 12 may also comprise an enclosed layer of water which is positioned atop of the PCM to provide for further support to the infant as well as to ensure that the heat and temperature is uniformly distributed over the assembly 12. The assembly 12 may be comprised of a retaining tray 40 which defines a PCM retaining portion 42 surrounded by a retaining edge or lip 46 around the periphery of the tray 40, as shown in the perspective view of FIG. 5A as well as the top and side views of FIGS. 5B and 5C. One or more temperature sensors 44, e.g., thermistor, thermocouple, etc., may be positioned along the retaining tray 40 to monitor the temperature of the PCM.

While the retaining tray 40 may be comprised of various materials, it may be fabricated from, e.g., PVC, and have a thickness of, e.g., 0.7 mm. Moreover, the tray 40 may have an overall length of, e.g., 415 mm, an overall width of, e.g., 245.5 mm, as shown in FIG. 5B, as well as a height of e.g., 25.4 mm, as shown in FIG. 5C. The dimensions provided are merely exemplary and may be varied depending upon the desired overall dimensions.

The retaining tray 40 may provide a retaining channel or depression for the temperature regulation element 50 which may enclose the PCM. The PCM may generally comprise a substance which melts or solidifies at a particular temperature or temperature range thereby storing or releasing energy at that particular temperature or range. During a phase change from liquid to solid or vice versa, the energy absorbed or released by the PCM is used to facilitate the phase change of the material and thus the temperature of the PCM remains constant. The PCM preferably has a high heat of fusion which allow it to store and release large amounts of energy. Additionally, the PCM may comprise either an organic or inorganic material, e.g., n-eicosane, Tetradecanol, eutectic mixtures, or any other suitable material.

While the temperature regulation element 50 may continue to provide heat at its operating temperature (e.g., for about 8 hours or more at an ambient temperature of about 25° C.), the heat-providing capacity of the temperature regulation element 50 may depend on factors such as weight and health of the newborn as well as operating conditions such as ambient temperature, as described in further detail below.

With the temperature regulation element 50 so positioned within retaining portion 42, as shown in FIG. 5D, a separate tray covering 52 may be secured to the retaining edge or lip 46 around the periphery of the tray 40 to fully enclose the temperature regulation element 50 within the tray 40. The tray covering 52, as shown in the top view of FIG. 5E, may be similarly fabricated from PVC with a thickness of 0.5 mm and similar dimensions to the retaining tray 40. Moreover, the tray covering 52 not only secures the temperature regulation element 50 within the assembly 12 but also maintains a flattened configuration of the element 50 and prevents the element 50 from forming a bulged or tear-drop shape.

With the temperature regulation element 50 fully enclosed, a separate water layer 56 retained within an enclosure may then be secured upon the tray covering 52, as shown in the top view of FIG. 5F. As described above, the water layer 56 may provide for further support to the infant as well as to ensure that the heat and temperature is uniformly distributed over the assembly 12. The interface attachment 54 may also be seen with wires extending from the temperature sensor 44 within the retaining tray 40. The control/indicator interface 14 may then be attached to the retaining tray 40 and electrically coupled to the temperature sensor 44 within, as shown in the perspective view of FIG. 5G.

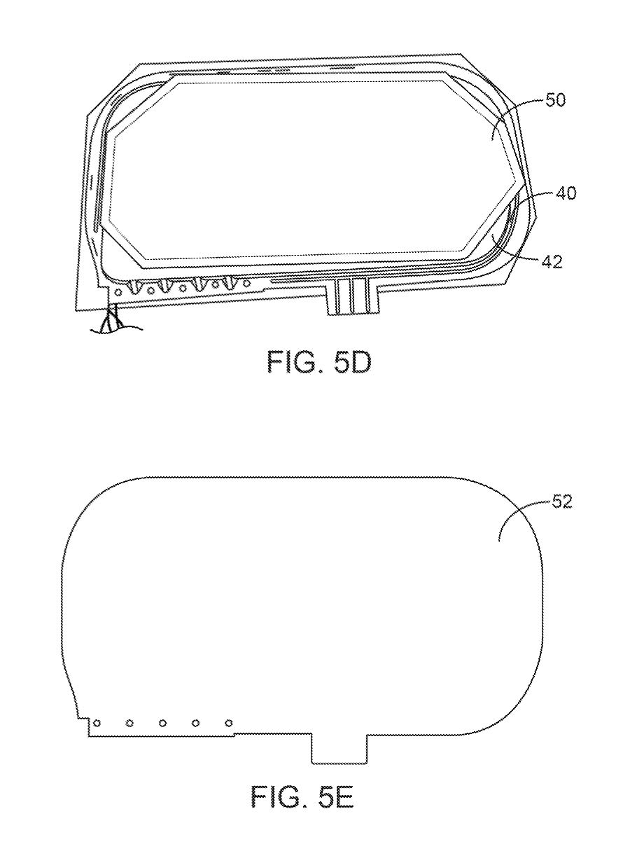

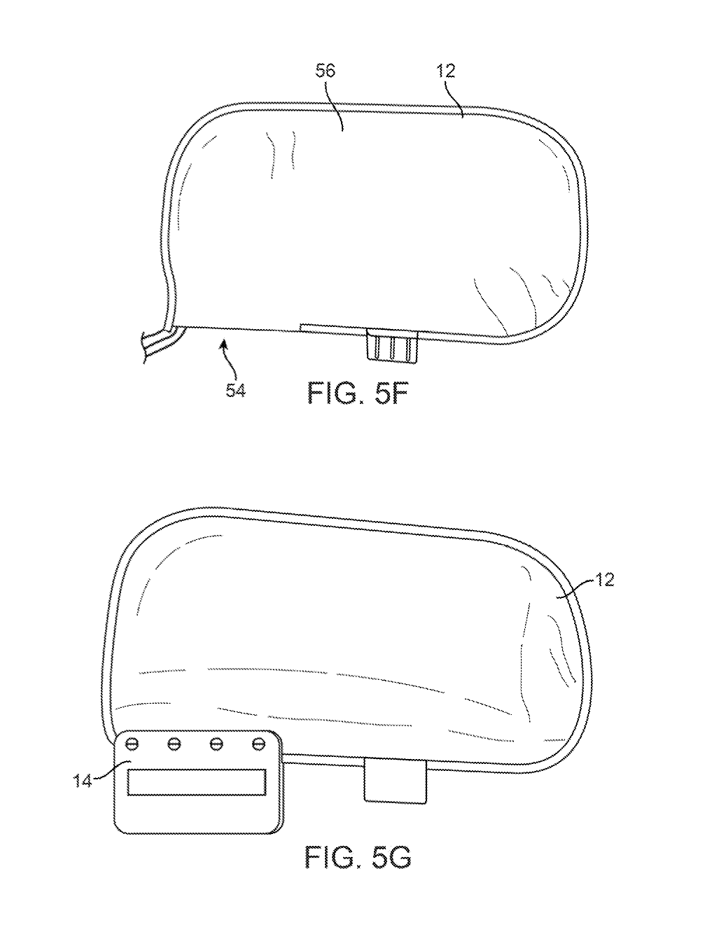

The fully assembled temperature regulation assembly 12 may accordingly be heated (and re-heated as needed) within the retaining pouch 36 of heating assembly 30 and then transferred to the retaining pouch 20 of bedding 16. FIG. 6 shows a perspective view of heating assembly 30 and FIG. 7 shows a partial cross-sectional top view of reservoir 60 which may be comprised of a plastic barrier film bag and placed within the heating assembly 30 for retaining the heated water. The reservoir 60 may comprise a port or spout 34 for introducing the heated water within the interior volume 64. Moreover, the reservoir 60 may further define a tapered edge 62 extending from the position of the port 34 to facilitate emptying of water from the reservoir 60 for storage or for re-filling of the reservoir 60 with more heated water.

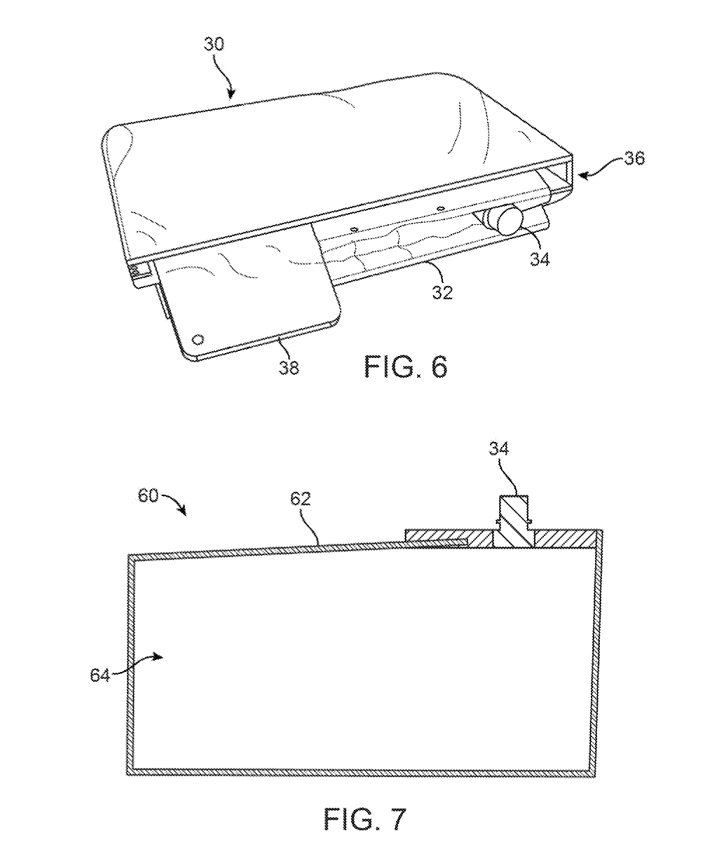

A top view of the temperature regulation assembly 12 placed within the heating assembly 30 is shown in FIG. 8 to illustrate how the control/indicator interface 14 may extend from the heating assembly 30 to enable the continued monitoring of its temperature during heating of the assembly temperature regulation assembly 12. Moreover, the heated water may be introduced into the reservoir 60 through port 34, if needed, while the temperature regulation assembly 12 is removed from or remains positioned within pouch 36.

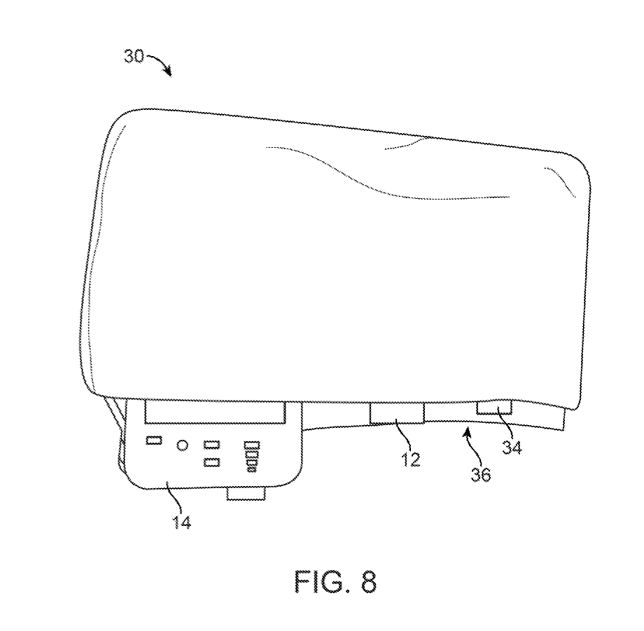

Once the temperature regulation assembly 12 is placed within bedding 16 for use with an infant, the PCM within temperature regulation element 50 may provide for warming heat for up to 8 hours or more. FIG. 9 shows a graph 70 illustrating the temperature change over time provided by the PCM where the heat released may be maintained within a desired operating temperature range between an initial temperature of, e.g., 41° C., down to a temperature of, e.g., 35° C., over a period of several hours while maintaining a relatively constant temperature variance.

Turning now to the control/indicator interface 14, the interface 14 may comprise a processor and associated electronics within which continuously monitor both the temperature of the temperature regulation assembly as well as the ambient temperature. In the event that the various temperatures are too high or too low for any reason, the interface 14 may provide a visual and/or auditory indicator or alarm to alert the user to take corrective action, if needed, such as when the temperature regulation assembly 12 is safe to use with an infant (e.g., a green light may flash with or without an auditory alarm) or when the temperature regulation assembly 12 is unsafe due to high or low temperatures (e.g., a red light may flash). The indicator 14 may be powered by an on-board power supply, e.g., batteries, or any other type of power supply.

The control/indicator interface 14 may also incorporate memory storage to collect and store various forms of data monitored and sensed by the system or user as well as a communication interface to transfer the data (e.g., wired or wirelessly) to or from an external device.

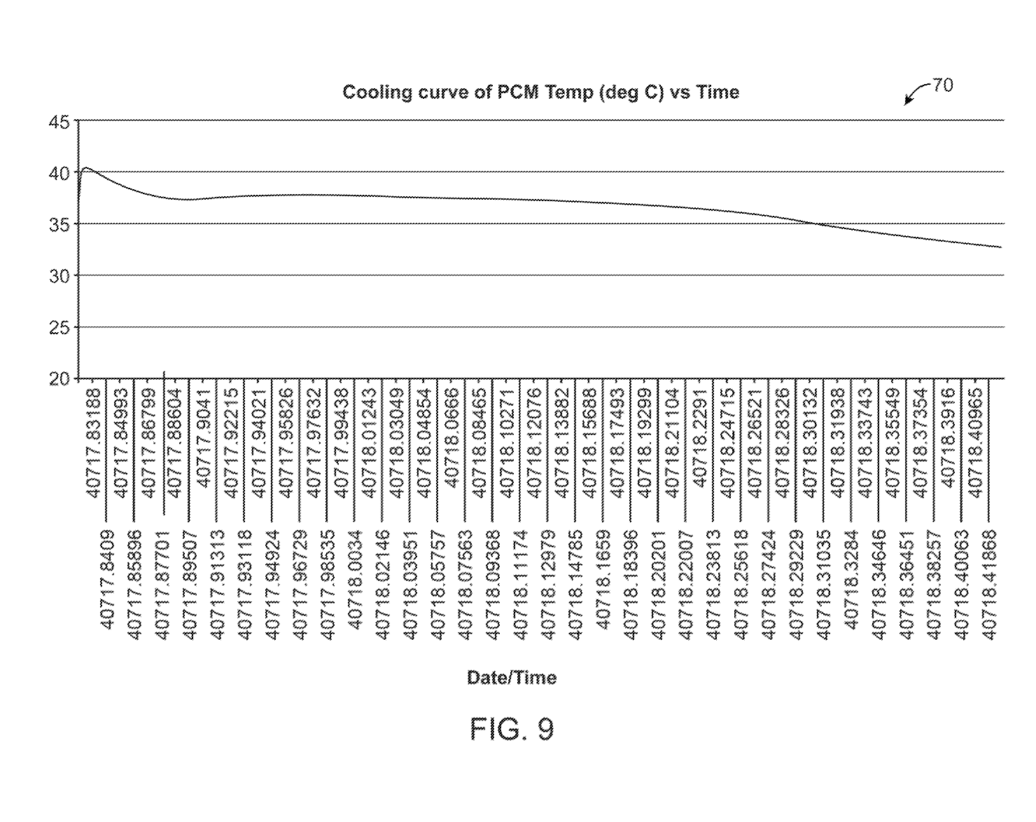

The indicator 14 may accordingly provide a number of different indications or alerts to the user. Some examples of the various indications or alerts which may be displayed or otherwise provided as shown in the chart 80 of FIG. 10. These indications are provided as examples and are not intended to limit the various types of indications or alerts which may be provided.

The indicator 14 may also be provided with one or more actuation features, such as a button, touchscreen, etc. to place the processor within the indicator within one of several available functions. A first function may silence or “snooze” any visible and/or audible alarms for about, e.g., 1.5 minutes, although this period of time may be programmed to be more or less. A second function of the indicator may be used when the assembly 12 is in use with an infant. When activated, a PCM charge indication feature may allow the user to estimate how long the assembly 12 will last so that the user may plan accordingly. Finally, a third function may initiate a demonstration mode after which the indicator 14 may revert to its normal state.

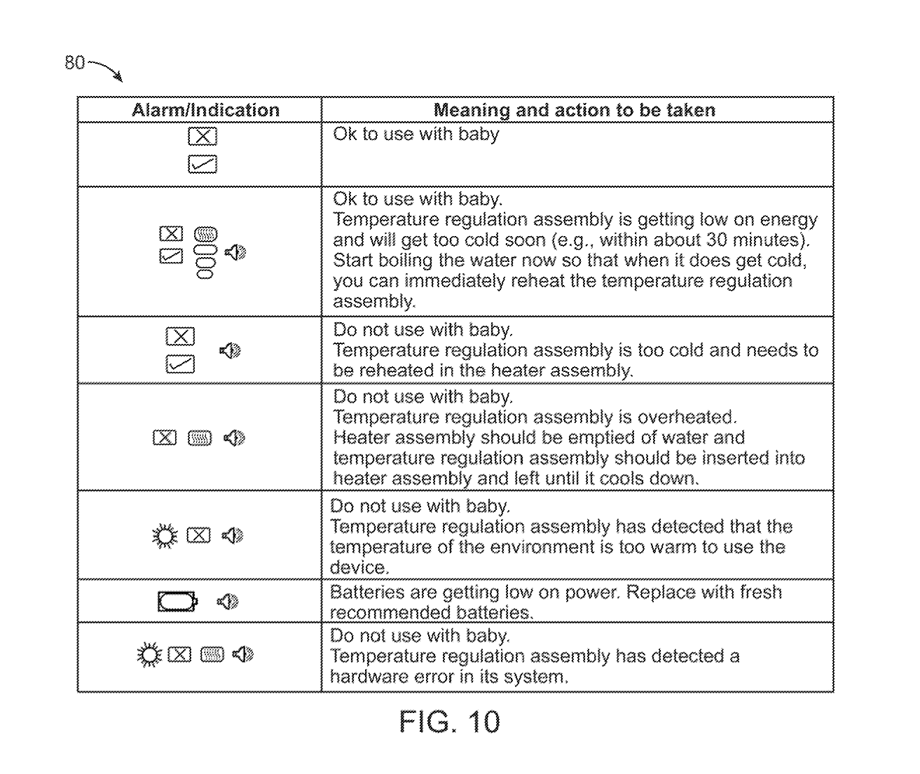

Because of the differences between the temperature of the assembly 12 and the variability of the ambient temperature, the processor within indicator 14 may be programmed to follow one of several different algorithms to ensure safety to the infant. An example is shown in the flow diagram of FIG. 11 illustrating the flow between the various states of the temperature regulation assembly 12 and corresponding indications for which the indicator 14 and processor may be programmed to monitor and/or provide indications or alerts to the user.

The indicator 14 may be programmed to distinguish when the assembly 12 is positioned inside the receiving pouch 36 of the heating assembly 30 (e.g., when the interface attachment 38 is coupled to the indicator 14) as shown by the various states within block 90. Similarly, the indicator 14 may also be programmed to distinguish when the assembly 12 is positioned outside of the heating assembly 30 such as when the unit is simply removed from the heating assembly 30 or when the unit is placed within the receiving pouch 20 of the bedding 16 for use as shown by the various states within block 92.

When the assembly 12 is positioned within the heating assembly 30, it may comply with one of several different states. For instance, prior to or during heating of the assembly 12, the assembly 12 may be indicated as being in a COLD state 94, e.g., by showing a red light and remaining silent, until its temperature is sensed by indicator 14 as being below 36.0° C. In the event its temperature rises to be greater than or equal to 36.5° C., indicator 14 may enter an OK TO USE state 96, e.g., by showing a green light and alerting with a Medium Priority alarm. However, if the assembly 12 becomes overheated and unsafe for use, indicator 14 may enter an OVERHEATED state 98, e.g., by showing a red light and alerting with a High Priority alarm (which may or may not be optionally silenced). The assembly 12 may be considered overheated if the sensed temperature is either greater than or equal to 41° C. or if the sensed temperature is greater than or equal to 38.5° C. along with a temperature rate change over time (dT/dt) of 0.2 degrees/30 seconds is also detected. However, if the temperature falls below 40.5° C. along with a temperature rate change over time of 0 degrees/minute, then the indicator 14 may revert to its OKAY TO USE state 96.

Because use of the assembly 12 may be affected by the ambient temperature, the indicator 14 may also be configured to sense the ambient temperature as well and account for this variable condition. For instance, in the event that a HIGH AMBIENT state 100 is detected (e.g., when the ambient temperature is greater than or equal to 32° C.), indicator 14 may show a red light yet remain silent if the assembly 12 is still positioned within the heating assembly 30. If the temperature of the assembly 12 is sensed to be greater than or equal to 41° C., then indicator 14 may enter the OVERHEATED state 98. Otherwise, if the ambient temperature is less than or equal to 32° C. and the detected ambient temperature remains so for greater than 2 minutes, then the indicator 14 may enter into a COLD state 94 or OK TO USE state. The 2 minute waiting period may be programmed into the indicator 14 to ensure that any temporary temperature fluctuations are dampened and accounted for although this waiting period may be less than or greater than 2 minutes or may be omitted entirely if so desired.

In the event that the temperature regulation assembly 12 has been removed from the heating assembly 30. e.g., for use within bedding 16, then the indicator 14 may indicate or alert any one of the states shown in block 92. For instance, in the event that a temperature of the assembly is less than 35° C., a COLD state 102 may be indicated, e.g., by showing a red light and indicating a High Priority alarm, to alert the user that the assembly 12 requires heating and should not be used with an infant.

However, if the assembly 12 is sensed to fall below 35.3° C. during its use, then an EARLY HEATING INDICATION state 104 may be indicated, e.g., by showing a green light and alerting with an optional repeating alert or alarm, to alert the user that the assembly 12 may need to be re-heated within a predetermined period of time, e.g., within 30 minutes, to allow the user to make preparations accordingly such as by heating water for the heating assembly 30. If the assembly 12 is sensed to further fall below 35.0° C., then it may re-enter the COLD state 102; otherwise, if the assembly 12 is sensed to rise to be greater than or equal to 36° C. then the indicator 14 may enter an OK TO USE state 106. However, in the event that the assembly 12 is sensed to rise in temperature to be greater than or equal to 41° C., then the indicator 14 may enter an OVERHEATED state 108, e.g., by showing a red light and alerting with a High Priority alarm, to alert the user that the assembly 12 should not be used with the infant and should be allowed to cool before use. If the indicator 14 senses that the temperature has fallen below 38.5° C., then the indicator 14 may enter back into its OK TO USE state 106.

As described above, because use of the assembly 12 may be affected by the ambient temperature as sensed by the indicator 14, an additional state may be accounted for as shown by the HIGH AMBIENT state 110. In the event that an ambient temperature of greater than or equal to 32° C. is detected over a waiting period of, e.g., 2 minutes, is sensed, then the indicator 14 may enter the HIGH AMBIENT state 110 from either its COLD state 102, EARLY HEATING INDICATION state 104, or from its OK TO USE state 106, e.g., by showing a red light and alerting a High Priority alarm, as an indication to the user that use of the assembly 12 may not be needed due to the already high ambient temperature. Otherwise, if the ambient temperature is sensed to be equal to or less than 32° C. over a period of, e.g., 2 minutes, then the indicator 14 may enter back into its COLD state 102. However, if the temperature of the assembly 12 is further sensed to be equal to or greater than 41° C., then the indicator 14 may further enter its OVERHEATED state 108 to further alert the user that either the assembly 12 may need to be first cooled and/or until the ambient temperature falls sufficiently.

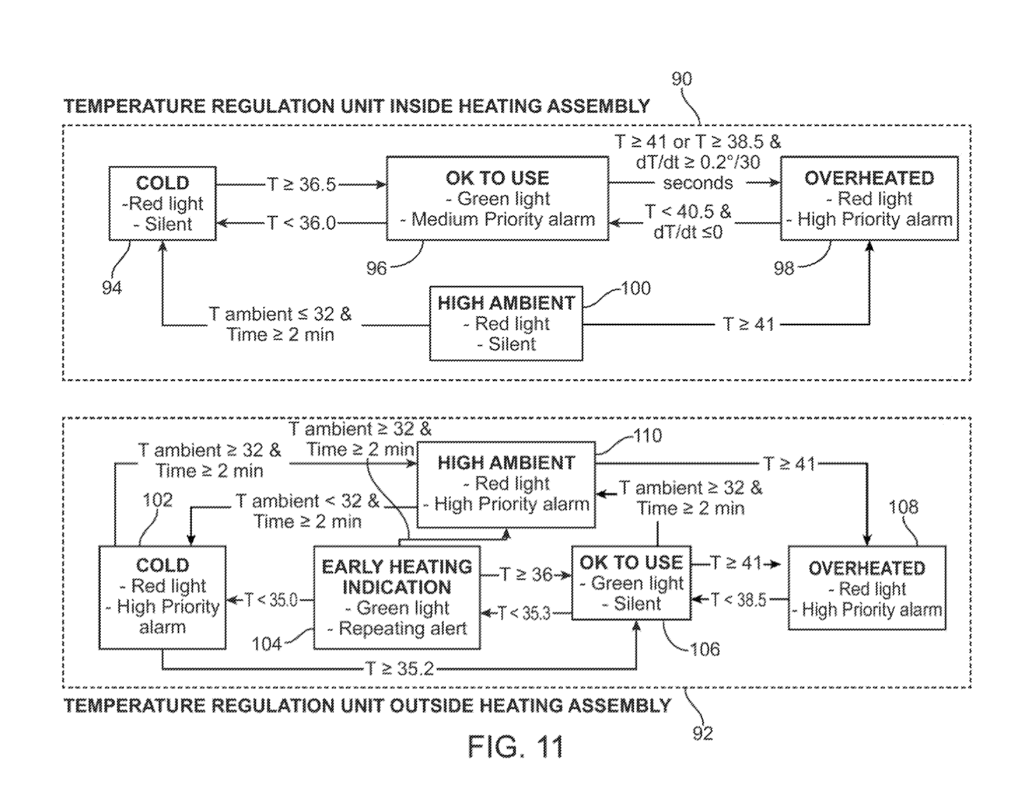

With the various states (when the assembly 12 is either within the heating assembly 30 or outside the heating assembly 30), FIG. 12 shows the flow diagram of FIG. 11 but with indications of when the temperature regulation assembly 12 may be re-positioned between the heating assembly 30 and bedding 16 (or simply removed from the beating assembly 30). For instance, when the indicator 14 shows the assembly 12 as being in a COLD state 94 or 102, the assembly 12 may be re-positioned between the heating assembly 30 or removed from the heating assembly 30. Similarly, if the indicator 14 shows assembly 12 as being in its EARLY HEATING INDICATION state 104, assembly 12 may be re-positioned into the heating assembly 30 where it may enter its COLD state 94 for further heating.

In the event that the indicator 14 shows that the assembly 12 is OKAY TO USE 96 within the heating assembly 30 or OKAY TO USE 106 when removed from the heating assembly 30, the assembly 12 may be re-positioned between either state. Similarly, in the event that the indicator 14 shows that the assembly 12 is OVERHEATED 98 within the heating assembly 30 or OVERHEATED 108 when removed from the heating assembly 30, the assembly 12 may be re-positioned between either state for cooling. Likewise, if a HIGH AMBIENT state 100 is indicated within the heating assembly 30 or HIGH AMBIENT state 110 when removed from the heating assembly 30, the assembly 12 may be re-positioned between either state until the ambient temperature falls.

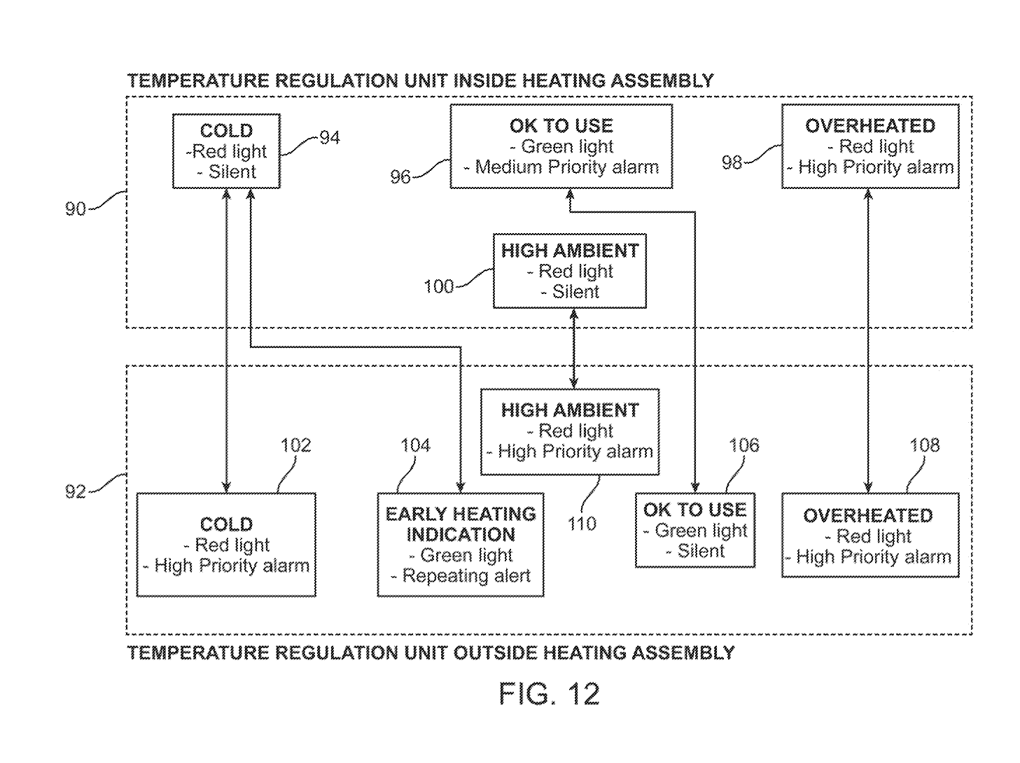

Additionally and/or alternatively, the system may also incorporate a number of features to further provide for the safety and comfort of the infant placed within the system. For instance, the control/interface unit 14 may alert to conditions such as the ambient temperature being too low or too high or it may automatically trigger other actions that ensure usage of the device when in a safe ambient temperature range. Moreover, the system may also incorporate one or several monitoring peripherals 120 to monitor various physiological parameters of the infant (e.g., vital parameters such as infant temperature, weight, oxygen levels, respiration, heart rate, etc.). FIG. 13 shows an example of an infant IF placed upon the bedding 16 of the system. A monitoring peripheral 120, which may be configured to sense any number of the physiological parameters of the infant IF, may be attached either directly upon or in proximity to the infant IF. Moreover, the monitoring peripheral 120 may be optionally coupled to the control/interface unit 14 (e.g., via a wire 122 or wirelessly) to transmit the physiological data to the control/interface unit 14 where the processor may utilize this information for monitoring or for storing the data to memory. Alternatively, the peripheral 120 may instead be in communication with an extemal unit.

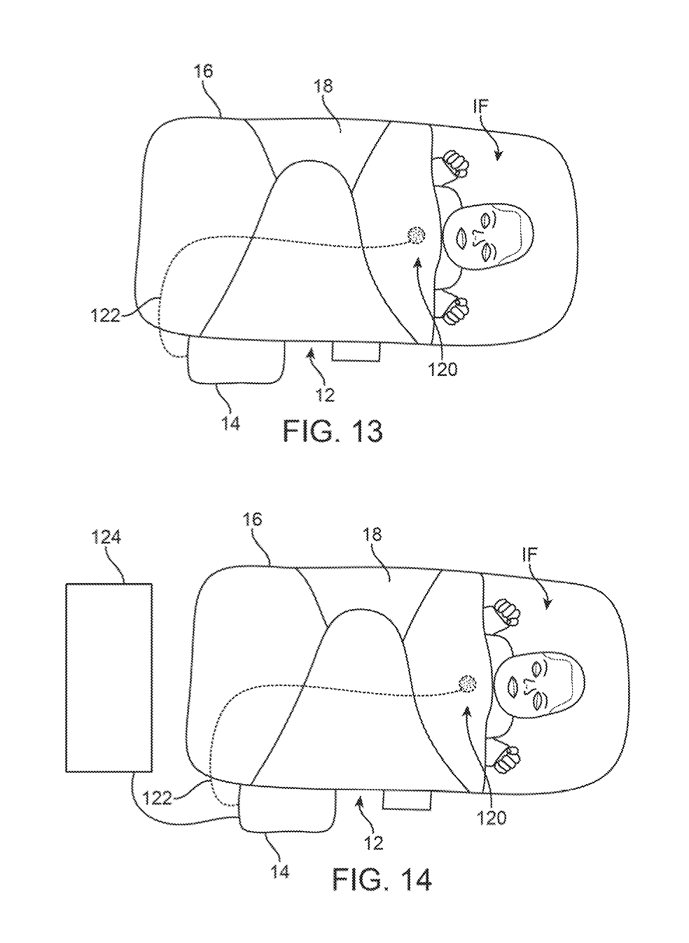

Additionally, one or more monitoring and/or peripheral devices 124, e.g., air-purifiers, may be used in combination with the system and optionally connected to the control/indicator interface 14 (e.g., wired or wirelessly), as shown in FIG. 14. Such devices 124 may be used to control or create a local environment or microclimate around or in proximity to the infant such as creating or maintaining hygienic levels of air quality (in one example) around the infant IF. Other devices may be used in other examples such as ambient warmers, humidity controllers, etc. Such monitoring peripherals 120 as well as monitoring and/or peripheral devices 124 may be used in any number of combinations with any of the variations described herein.

While illustrative examples are described above, it will be apparent to one skilled in the art that various changes and modifications may be made therein. Moreover, various apparatus or procedures described above are also intended to be utilized in combination with one another, as practicable. The appended claims are intended to cover all such changes and modifications that fall within the true spirit and scope of the invention.

What is claimed is:

1. A temperature regulation assembly for regulating temperature of an infant, comprising:
a phase change material which changes between a liquid phase and a solid phase within a predetermined temperature range;
at least one temperature sensor in thermal communication with the phase change material; and
a control/indicator interface in communication with the at least one temperature sensor, the interface being programmed to provide an indication or alert to a user when the phase change material is within the predetermined temperature range.

2. The assembly of claim 1 further comprising a retaining tray within which the phase change material is retained.

3. The assembly of claim 2 wherein the phase change material is retained in a flattened configuration within the retaining tray.

4. The assembly of claim 1 wherein the at least one temperature sensor comprises a thermistor or thermocouple adjacent to the phase change material.

5. The assembly of claim 1 wherein the control/indicator interface is programmed to provide the indication or alert when the phase change material is within the predetermined temperature range of between 35° to 41° C.

6. The assembly of claim 1 further comprising a water layer adjacent to the temperature regulation assembly.

7. The assembly of claim 1 further comprising a heating assembly having a fluid reservoir in thermal communication with a retaining pouch configured to receive the temperature regulation assembly.

8. The assembly of claim 7 wherein the fluid reservoir is sized to receive 2.7 to 3 liters of fluid.

9. The assembly of claim 7 wherein the control/indicator interface is programmed to provide the indication or alert when the temperature regulation assembly is within the retaining pouch and is greater than or equal to 36.5° C.

10. The assembly of claim 7 wherein the control/indicator interface is programmed to provide an indication or alert for cooling the phase change material when the temperature regulation assembly is within the retaining pouch and when the temperature regulation assembly is above 38.5° C. with a temperature rate change of 0.2 degrees/30 seconds or greater than 41° C.

11. The assembly of claim 7 wherein the control/indicator interface is programmed to provide an indication or alert for ceasing use of the phase change material when the temperature regulation assembly is within the retaining pouch and an ambient temperature is above 32° C.

12. The assembly of claim 1 wherein the control/indicator interface is programmed to provide the indication or alert when the temperature regulation assembly is less than or equal to 35° C.

13. The assembly of claim 1 wherein the control/indicator interface is programmed to provide an indication or alert for cooling the phase change material when the temperature regulation assembly is above 41° C.

14. The assembly of claim 1 wherein the control/indicator interface is programmed to provide an indication or alert for ceasing use of the phase change material when an ambient temperature is above 32° C.

15. The assembly of claim 1 further comprising at least one monitoring peripheral configured to be placed upon or in proximity to the infant, where the at least one monitoring peripheral is in communication with the control/indicator interface.

16. The assembly of claim 1 further comprising at least one monitoring and/or peripheral device in proximity to the infant, where the at least one monitoring and/or peripheral device is in communication with the control/indicator interface.

17. A method of regulating temperature of an infant, comprising:
providing a temperature regulation assembly having a phase change material which changes between a liquid phase and a solid phase within a predetermined temperature range;
positioning the temperature regulation assembly within a retaining pouch of a heating assembly having an adjacent reservoir filled with a heated volume of water;
providing an indication or alert when a temperature of the temperature regulation assembly is between 35° to 41° C.; and
further positioning the temperature regulation assembly within a retaining pouch of a bedding such that the temperature of the infant in contact with the bedding is regulated.

18. The method of claim 17 wherein the temperature regulation assembly further comprises a retaining tray within which the phase change material is retained.

19. The method of claim 17 wherein positioning the temperature regulation assembly further comprises monitoring a temperature of the temperature regulation assembly via at least one temperature sensor in thermal contact with the phase change material.

20. The method of claim 19 wherein monitoring a temperature comprises monitoring via a control/indicator interface having a processor.

21. The method of claim 17 wherein providing an indication or alert further comprises providing the indication or alert when the phase change material is within the predetermined temperature range of between 35° to 41° C.

22. The method of claim 17 wherein providing an indication or alert further comprises providing the indication or alert when the temperature regulation assembly is greater than or equal to 36.5° C.

23. The method of claim 17 wherein providing an indication or alert further comprises providing the indication or alert when the temperature regulation assembly above 38.5° C. with a temperature rate change of 0.2 degrees/30 seconds or greater than 41° C.

24. The method of claim 17 wherein providing an indication or alert further comprises providing the indication or alert when an ambient temperature is above 32° C.

25. The method of claim 17 wherein providing an indication or alert further comprises providing the indication or alert when the temperature regulation assembly is less than or equal to 35° C.

26. The method of claim 17 wherein providing an indication or alert further comprises providing the indication or alert when the temperature regulation assembly is above 41° C.

27. The method of claim 17 further comprising monitoring one or more physiological parameters of the infant via at least one monitoring peripheral configured to be placed upon or in proximity to the infant.

28. The method of claim 17 further comprising controlling a local environment of the infant via at least one monitoring and/or peripheral device in proximity to the infant.
